(19) United States
(12) Patent Application Publication
(10) Pub. No.: US 2010/0057118 A1
(43) Pub. Date: Mar. 4, 2010

(54) ULTRASONIC SURGICAL BLADE

(76) Inventors: Timothy G. Dietz, Terrace Park, OH (US); Kevin L. Houser, Springboro, OH (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/540,573

(22) Filed: Aug. 13, 2009

Related U.S. Application Data
(60) Provisional application No. 61/093,836, filed on Sep. 3, 2008.

(51) Int. Cl. A61B 17/32

(52) U.S. Cl. 606/169

(57) ABSTRACT

An ultrasonic surgical blade includes a body having an external surface, at least one cutting edge, and a distal end. In at least one embodiment, the cutting edge can be defined by first and second surfaces which define an angle therebetween. In various embodiments, at least a portion of the cutting edge comprises a sharp point. In other embodiments, at least a portion of the cutting edge and surface comprise a sharp point and or a beveled surface.

### CROSS REFERENCE TO RELATED APPLICATIONS

This present application claims the benefit of U.S. Provisional application Ser. No. 61/093,836, filed on Sep. 3, 2008, the contents of which are incorporated by reference herein.

### STATEMENT REGARDING FEDERALLY SPONSORED RESEARCH OR DEVELOPMENT

Not Applicable.

### FIELD OF THE INVENTION

The various embodiments relate, in general, to ultrasonic surgical blades for use in surgical instruments and, more particularly, to an ultrasonic surgical blade with improved cutting and coagulation features.

### BACKGROUND OF THE INVENTION

Ultrasonic instruments, including both hollow core and solid core instruments, are used for the safe and effective treatment of many medical conditions. Ultrasonic instruments, and particularly solid core ultrasonic instruments, are advantageous because they may be used to cut and/or coagulate organic tissue using energy in the form of mechanical vibrations transmitted to a surgical end effector at ultrasonic frequencies. Ultrasonic vibrations, when transmitted to organic tissue at suitable energy levels and using a suitable end effector, may be used to cut, dissect, elevate or cauterize tissue or to separate muscle tissue off bone. Ultrasonic instruments utilizing solid core technology are particularly advantageous because of the amount of ultrasonic energy that may be transmitted from the ultrasonic transducer, through a waveguide, to the surgical end effector. Such instruments may be used for open procedures or minimally invasive procedures, such as endoscopic or laparoscopic procedures, wherein the end effector is passed through a trocar to reach the surgical site.

Activating or exciting the end effector (e.g., cutting blade) of such instruments at ultrasonic frequencies induces longitudinal vibratory movement that generates localized heat within adjacent tissue, facilitating both cutting and coagulation. Because of the nature of ultrasonic instruments, a particular ultrasonically actuated end effector may be designed to perform numerous functions, including, for example, cutting and coagulation.

Ultrasonic vibration is induced in the surgical end effector by electrically exciting a transducer, for example. The transducer may be constructed of one or more piezoelectric or magnetostrictive elements in the instrument hand piece. Vibrations generated by the transducer section are transmitted to the surgical end effector via an ultrasonic waveguide extending from the transducer section to the surgical end effector. The waveguides and end effectors are designed to resonate at the same frequency as the transducer. Therefore, when an end effector is attached to a transducer the overall system frequency is the same frequency as the transducer itself.

The shape of an ultrasonic surgical blade or end-effector used in an ultrasonic surgical instrument can define at least four important aspects of the instrument. These are: (1) the visibility of the end-effector and its relative position in the surgical field, (2) the ability of the end-effector to access or approach targeted tissue, (3) the manner in which ultrasonic energy is coupled to tissue for cutting and coagulation, and (4) the manner in which tissue can be manipulated with the ultrasonically inactive end-effector. It would be advantageous to provide an improved ultrasonic surgical instrument blade or end-effector optimizing at least these four aspects of the instrument.

Solid core ultrasonic surgical instruments may be divided into two types, single element end effector devices and multiple-element end effector. Single element end effector devices include instruments such as scalpels, and ball coagulators. Single-element end effector instruments have limited ability to apply blade-to-tissue pressure when the tissue is soft and loosely supported. Substantial pressure may be necessary to effectively couple ultrasonic energy to the tissue. This inability to grasp the tissue results in a further inability to fully coapt tissue surfaces while applying ultrasonic energy, leading to less-than-desired hemostasis and tissue joining. The use of multiple-element end effectors such as clamping coagulators includes a mechanism to press tissue against an ultrasonic blade that can overcome these deficiencies.

Ultrasonic clamp coagulators provide an improved ultrasonic surgical instrument for cutting/coagulating tissue, particularly loose and unsupported tissue, wherein the ultrasonic blade is employed in conjunction with a clamp for applying a compressive or biasing force to the tissue, whereby faster coagulation and cutting of the tissue, with less attenuation of blade motion, are achieved.

Current ultrasonic blade designs are optimized for soft tissues. The current blade designs, in the presence of a continuum of soft tissue, such as viscera, to tough tissue, such as cartilage, preferentially cut the soft tissues. When the blade hits harder or tougher tissue, the blade tends to deflect away from such tissue and continue along the path of least resistance. This performance is often preferred for dissecting between planes of tissue, but makes it difficult to cut tough tissue, such as cartilage.

Consequently, a significant need exists for an ultrasonic blade that is able to cut different tissue types. The present invention provides for such an ultrasonic device.

### BRIEF DESCRIPTION OF THE FIGURES

The novel features of the invention are set forth with particularity in the appended claims. The invention itself, however, both as to organization and methods of operation, may best be understood by reference to the following description, taken in conjunction with the accompanying drawings in which:

FIG. 1 illustrates one embodiment of an ultrasonic surgical system;

FIG. 2 is a perspective view of a prior art ultrasonic blade;

FIG. 3 is a perspective view of one embodiment of the present invention;

FIG. 4 is a perspective view of an alternate expression of on embodiment of the present invention;

FIG. 5 is an elevation and partial cut away view of one expression of an embodiment of the invention;

FIG. 6 is an elevation and partial cut away view of an alternate expression of an embodiment of the invention;

FIG. 7 is an elevation and partial cut away view of an alternate expression of an embodiment of the invention;

FIG. 8 is a partial plan view of an alternate expression of an embodiment of the invention;

FIG. 9 is a perspective view of an alternate embodiment of the invention illustrating proximal serrations;

FIG. 10 is a plan view of an alternate embodiment of the invention; and

FIG. 11 is a plan view of an alternate embodiment of the invention.

### DETAILED DESCRIPTION OF THE INVENTION

Before explaining the present invention in detail, it should be noted that the embodiments are not limited in its application or use to the details of construction and arrangement of parts illustrated in the accompanying drawings and description. The illustrative embodiments may be implemented or incorporated in other embodiments, variations and modifications, and may be practiced or carried out in various ways. For example, the surgical instruments and blade configurations disclosed below are illustrative only and not meant to limit the scope or application thereof. Further, unless otherwise indicated, the terms and expressions employed herein have been chosen for the purpose of describing the illustrative embodiments for the convenience of the reader and are not to limit the scope thereof.

The various embodiments relate, in general, to ultrasonic surgical blades for use in surgical instruments and, more particularly, to an ultrasonic surgical blade with improved cutting and coagulation features. A blade according to various embodiments is of particular benefit, among others, in orthopedic procedures wherein it is desirable to remove tough tissue, such as cartilage, and/or tissue while controlling bleeding for removing muscle tissue from bone, due to its cutting and coagulation characteristics. A blade according to the various embodiments may reduce the user force required to remove muscle from bone and, in one embodiment, may be useful to simultaneously hemostatically seal or cauterize the tissue. Reducing the force to operate the surgical instrument may reduce user fatigue, improve precision and reduce unwanted tissue damage.

The blade, however, may also be useful for general soft tissue cutting and coagulation, for example in plastic surgeries, such as breast augmentation or reduction. Soft tissues are often difficult for the surgeon to suspend and tension so that ultrasonic tool pressure can be effectively applied to achieve efficient cutting action. The various embodiments of the invention allow the surgeon to capture and effectively tension the tissue against the cutting edges of the blade.

A blade according to various embodiments may be useful in spine surgery, especially to assist in posterior access in removing muscle from bone. A variety of different blade configurations are disclosed which may be useful for both open and laparoscopic applications.

Examples of ultrasonic surgical instruments are disclosed in U.S. Pat. Nos. 5,322,055 and 5,954,736 and in combination with ultrasonic blades and surgical instruments disclosed in U.S. Pat. Nos. 6,278,218B1, 6,283,981 B1, 6,309,400 B2, 6,325,811 B1 and 6,423,082 B1, for example, and commonly-owned, co-pending U.S. patent application Ser. No. 11/726,625, entitled Ultrasonic Surgical Instruments, filed on Mar. 22, 2007 and Ser. No. 11/998,543, entitled Ultrasonic Surgical Instrument Blades, filed on Nov. 30, 2007.

Certain exemplary embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting exemplary embodiments and that the scope of the various embodiments is defined solely by the claims. Further, it is understood that any one or more of the following-described embodiments, expressions of embodiments, examples, etc. can be combined with any one or more of the other following-described embodiments, expressions of embodiments, examples, etc. Such modifications and variations are intended to be included within the scope of the claims.

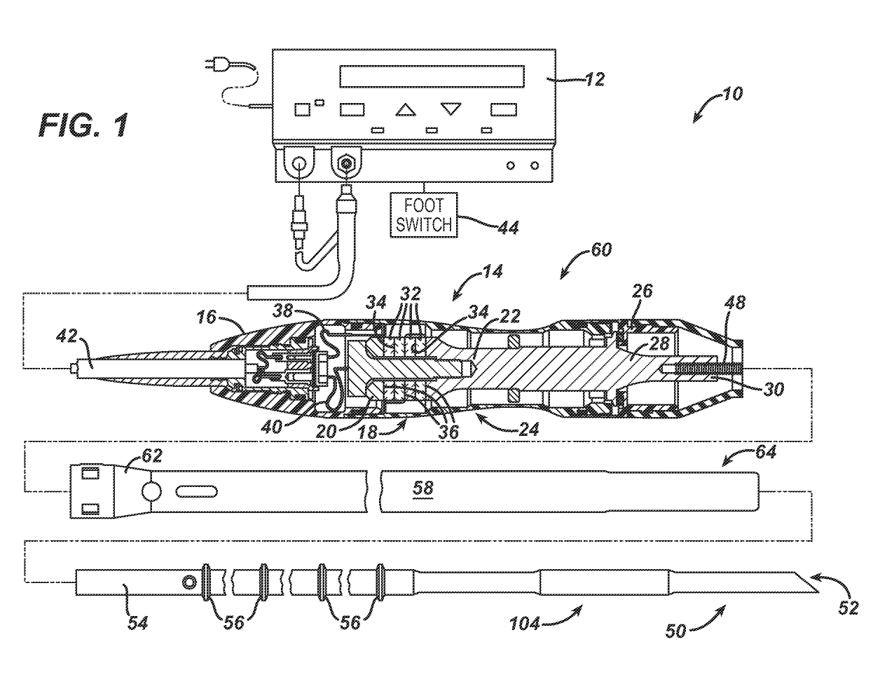

FIG. 1 illustrates one representative embodiment of an ultrasonic system 10. One embodiment of the ultrasonic system 10 comprises an ultrasonic signal generator 12 coupled to an ultrasonic transducer 14, a hand piece assembly 60 comprising a hand piece housing 16, and an end effector 50. The ultrasonic transducer 14, which is known as a “Langevin stack”, generally includes a transduction portion 18, a first resonator or end-bell 20, and a second resonator or fore-bell 22, and ancillary components. The ultrasonic transducer 14 is preferably an integral number of one-half system wavelengths (nλ/2) in length as will be described in more detail later. An acoustic assembly 24 includes the ultrasonic transducer 14, a mount 26, a velocity transformer 28, and a surface 30. The construction of such an ultrasonic system 10 is well documented in at least the references previously incorporated by reference and well known to one skilled in the art so its details will be left to such documentation and will not be expanded upon here.

It will be appreciated that the terms “proximal” and “distal” are used herein with reference to a clinician gripping the hand piece assembly 60. Thus, the end effector 50 is distal with respect to the more proximal hand piece assembly 60. It will be further appreciated that, for convenience and clarity, spatial terms such as “top” and “bottom” also are used herein with respect to the clinician gripping the hand piece assembly 60. However, surgical instruments are used in many orientations and positions, and these terms are not intended to be limiting and absolute.

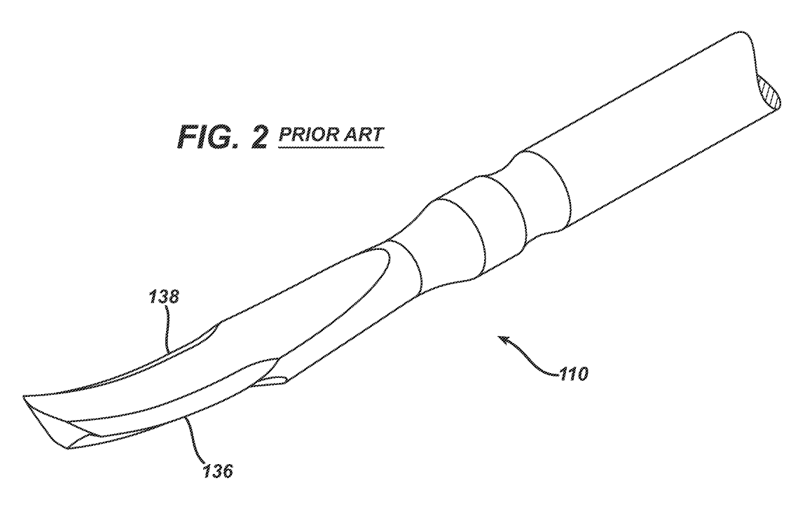

With reference to FIG. 2, the invention will be described in accordance with the ultrasonic blade disclosed in U.S. Pat. No. 6,423,082 B1 as exemplary only, and is not intending to be limiting and absolute. U.S. Pat. No. 6,423,082 discloses an ultrasonic surgical blade 110 including a top surface, a bottom surface and a cutting-edge 136. Blade 110 is a balanced curve blade wherein its longitudinal motion is greater than 97% of its overall motion (including transverse motion). The cutting-edge 136 is defined by a cutting-surface intermediate the top surface and the bottom surface, and the top surface has a width greater than the width of the bottom surface. The blade may be straight or curved. In one embodiment of the invention, at least a portion of the cutting-surface is substantially parallel to at least a portion of the top surface. In still another embodiment of the invention first and second side-walls intersect the top surface to form first and second cutting-edges that may be sharp or blunt. Alternately, a second cutting-edge 138 may be defined by a second cutting surface intermediate the top and bottom surfaces. Depending on the angle between the intermediate cutting-surface and the top surface, the cutting-edge may be sharp or blunt.

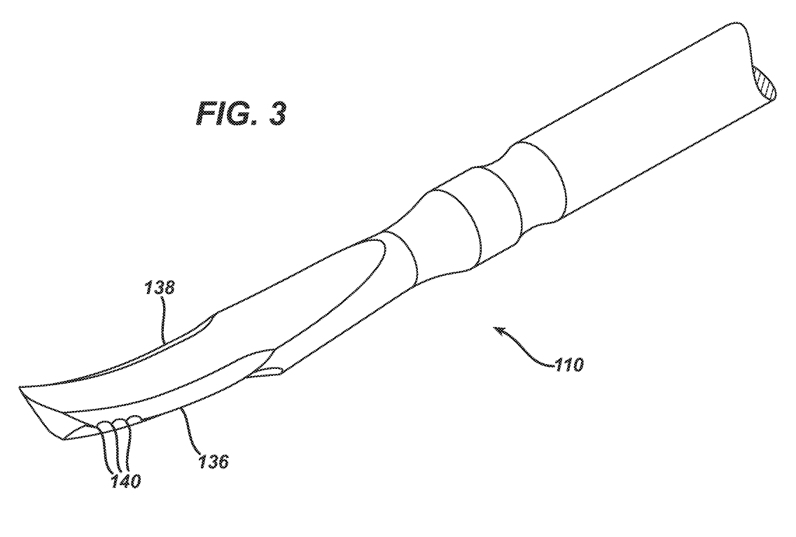

With reference to FIG. 3, shown is a first expression of a first embodiment of the current invention. The prior art blade of FIG. 2 is modified to present a plurality of sharp points 140 on sharp edge 136. Sharp points 140 create high pressure contact points with the tissue as the blade is pressed into or against the tissue. Sharp points 140 are defined by a cut radius, which in turn determine the spacing between adjacent sharp points. A small cut radius defines more sharp points 140 within a given distance and a larger cut radius defines fewer sharp points 140 within a given distance. The cut radius may vary from about 0.010 inches to about 0.060 inches. Alternatively, the cut radius may vary from one sharp point 140 to another.

Sharp points 140 initiate the dissection line allowing the user to cut with precision regardless of the tissue type. Sharp points 140 may exist on one sharp edge 136, or, alternatively, sharp points 140 may exist on both sharp edges 136 and 138. Further, sharp points 140 are shown at the distal end of ultrasonic surgical blade 110; however, blade 110 may be modified to include sharp points 140 at the proximal end or mid section of sharp edge 136 and/or 138. Further, sharp points 140 may exist along the entire length of sharp edge 136 and/or 138.

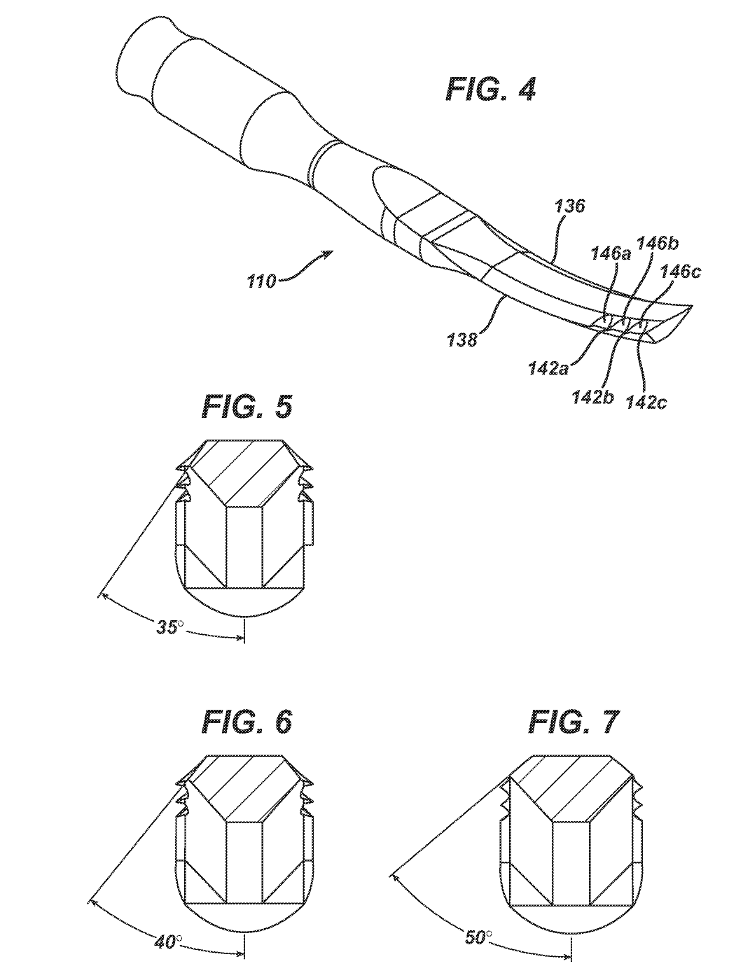

Referring now to FIGS. 4-7, shown is a second expression of the first embodiment of the invention. Shown are sharp points 142a-c in conjunction with beveled cutting edges 146a-c. Beveled cutting edges are defined by the angle of the bevel relative to the normal of blade 110 and the cut radius of the bevel. As would be appreciated by those skilled in the art, the bevel angle may be from greater than 0° to 90°. The embodiment of FIG. 3 illustrate sharp points without a bevel, therefore, the bevel angle is 0°. Preferably, in this expression of the embodiment, the bevel angle is from about 35° to about 50° and more preferably, 40°. The cut radius may vary from between about 0.010 inches to about 0.060 inches, and more preferably from about 0.020 inches to about 0.050 inches.

As shown in FIG. 4, sharp point 142a is common between bevel cutting edges 146a and 146b; sharp point 142b is common between bevel cutting edges 146b and 146c; and sharp point 142c is common only to bevel cutting edge 146c. Bevel cutting edges 146a-c may exist on one sharp edge 136, or, alternatively, bevel cutting edges 146a-c may exist on both sharp edges 136 and 138. Further, bevel cutting edges 146a-c are shown at the distal end of ultrasonic surgical blade 110; however, blade 110 may be modified to include bevel cutting edges 146a-c at the proximal end or mid section of sharp edge 136 and/or 138. Further, bevel cutting edges 146a-c may exist along the entire length of sharp edge 136 and/or 138. Further, the quantity of sharp points 142a-c and bevel cutting edges 146a-c are shown for illustrative purposes only, and is not intended to be limit in any fashion the scope of the invention. Unexpectedly, the inventor found enhanced performance of a blade comprising both sharp points and beveled cutting edges for cutting both soft and tough tissue. These blades showed enhanced cutting efficiency with respect to blades that were identical, but lacked the sharp points and the bevels. In soft tissues, such as fat and skin, the sharp points and/or the bevel features enable application of tension directly to the tissue via the blade geometry, rather than relying solely on secondary tensioning of the tissue. In cartilage, the points allowed the blade to initiate the incision and the beveled edges to complete the cut.

In an alternate expression of the current expression, sharp points 142a-c and bevel cutting edges 146a-c have a hardened surface coating. Such a coating may be that as disclosed in commonly-owned, co-pending U.S. Provisional Patent application, entitled Ultrasonic Surgical Blades, filed on Nov. 30, 2007 as Ser. No. 61/004,961, the contents of which are incorporated by reference herein, in its entirety.

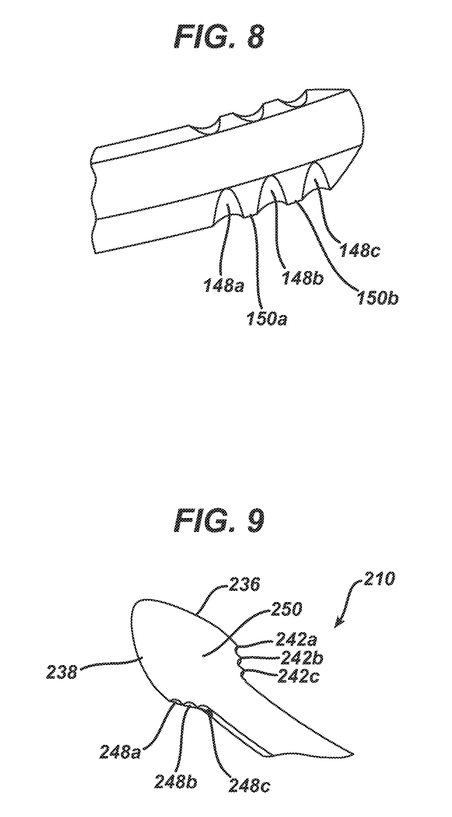

Referring now to FIG. 8, shown is a third expression of the first embodiment, where beveled cutting edges do not share a sharp point, but rather, beveled cutting edges 148a-c are separated by a cutting edge distance 150a-b. Cutting edge distance 150a-b varies depending upon the application, but in one exemplary embodiment, cutting edge distance 150a-c is from about 0.001 inches to about 0.10 inches. Further, cutting edge distance 150a-c do not have to be constant, and may vary in distance.

Beveled cutting edges 148a-c and cutting edge distance 150a-b may exist on one sharp edge 136, or, alternatively, beveled cutting edges 148a-c and cutting edge distance 150a-c may exist on both sharp edges 136 and 138. Further, beveled cutting edges 148a-c and cutting edge distance 150a-b are shown at the distal end of ultrasonic surgical blade 110; however, blade 110 may be modified to include beveled cutting edges 148a-c and cutting edge distance 150a-b at the proximal end or mid section of sharp edge 136 and/or 138. Further, beveled cutting edges 148a-c and cutting edge distance 150a-b may exist along the entire length of sharp edge 136 and/or 138.

FIG. 9 discloses an alternate embodiment of the present invention. Ultrasonic surgical blade 210 discloses an end effector 250 including a top surface, a bottom surface and cutting-edges 236 and 238. End effector 250 defines a spoon-like shape having a narrow width dimension at its distal end, a narrow width at its proximal end and intermediate the proximal end and distal end a width greater than the width dimensions at either the proximal or distal ends.

Shown are sharp points 242a-c in conjunction with a beveled cutting edges 248a-c. Beveled cutting edges are defined by the angle of the bevel relative to the normal of blade 210 and the cut radius of the bevel. As would be appreciated by those skilled in the art, the bevel angle may be from greater than 0° to 90°. The embodiment of FIG. 9 illustrate sharp points without a bevel, therefore, the bevel angle is 0°. Alternatively, the bevel angle may be from about 35° to about 50°. The cut radius may vary from between about 0.010 inches to about 0.060 inches, and more preferably from about 0.020 inches to about 0.050 inches.

Beveled cutting edges 248a-c may exist on one sharp edge 236, or, alternatively, beveled cutting edges 248a-c may exist on both sharp edges 236 and 238. Further, beveled cutting edges 248a-c are shown at the proximal end of ultrasonic end effector 250; however, end effector 250 may be modified to include beveled cutting edges 248a-c at the distal end or mid section of sharp edge 236 and/or 238. Further, beveled cutting edges 248a-c may exist along the entire length of sharp edge 236 and/or 238.

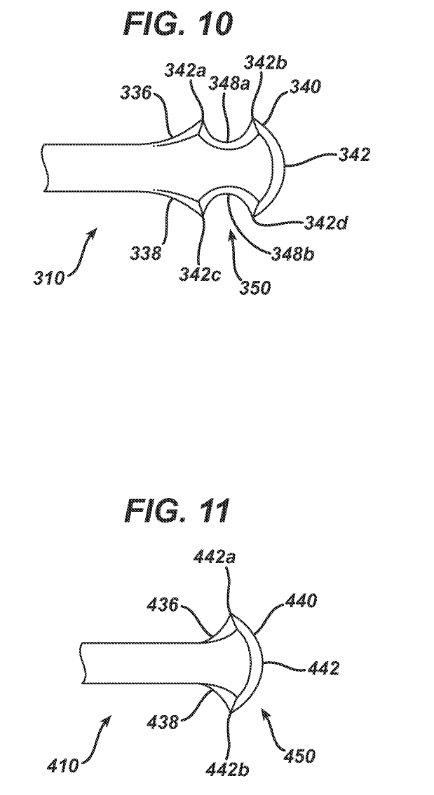

FIG. 10 illustrates an alternate embodiment of the present invention. Ultrasonic surgical blade 310 discloses an end effector 350 including a top surface, a bottom surface and cutting-edges 336 and 338. End effector 350 defines an arcuate distal end 340 with a cutting edge 342. Intermediate distal end 340 and cutting edges 336 and 338 are beveled cutting edges 348a-b and sharp points 342a-d. Beveled cutting edges are defined by the angle of the bevel relative to the normal of blade 310 and the cut radius of the bevel. As would be appreciated by those skilled in the art, the bevel angle may be from 0° to 90°.

FIG. 11 illustrates an alternate embodiment of the present invention. Ultrasonic surgical blade 410 discloses an end effector 450 including a top surface, a bottom surface and cutting-edges 436 and 438. End effector 450 defines an arcuate distal end 440 with a cutting edge 442. Intermediate cutting edge 440 and cutting edges 436 and 438 are sharp points 442a-b.

While the present invention has been illustrated by description of several embodiments, it is not the intention of the applicant to restrict or limit the spirit and scope of the appended claims to such detail. Numerous variations, changes, and substitutions will occur to those skilled in the art without departing from the scope of the invention. Moreover, the structure of each element associated with the present invention can be alternatively described as a means for providing the function performed by the element. Accordingly, it is intended that the invention be limited only by the spirit and scope of the appended claims.

What is claimed is:

1. An ultrasonic surgical blade, comprising:
a. a body having an external surface, a distal end movable relative to a longitudinal axis by vibrations applied thereto, and a cutting edge having a distal portion and a proximal portion, wherein at least a portion of the cutting edge comprises a sharp point.

2. The ultrasonic surgical blade of claim 1, wherein the sharp point is located at the distal portion.

3. The ultrasonic surgical blade of claim 1, wherein the sharp point is located at the proximal portion.

4. The ultrasonic surgical blade of claim 1, wherein the blade comprises a second cutting edge having a distal portion and proximal portion, wherein at least a portion of the second cutting edge comprises a sharp point.

5. The ultrasonic surgical blade of claim 1, wherein the cutting edge is straight.

6. The ultrasonic surgical blade of claim 1, wherein the cutting edge is curved.

7. The ultrasonic surgical blade of claim 1, wherein the cutting edge is defined by first and second surfaces which define an angle therebetween.

8. The ultrasonic surgical blade of claim 1, wherein the distal end defines a beveled cutting surface.

9. An ultrasonic surgical blade, comprising:
a. a body having an external surface, a distal end movable relative to a longitudinal axis by vibrations applied thereto, and a cutting edge having a distal portion and a proximal portion, wherein at least a portion of the cutting edge comprises a beveled surface.

10. The ultrasonic surgical blade of claim 9, wherein the beveled surface is located at the distal portion.

11. The ultrasonic surgical blade of claim 9, wherein the beveled surface is located at the proximal portion.

12. The ultrasonic surgical blade of claim 9, wherein the blade comprises a second cutting edge having a distal portion and proximal portion, wherein at least a portion of the second cutting edge comprises a beveled surface.

13. The ultrasonic surgical blade of claim 9, wherein the cutting edge is straight.

14. The ultrasonic surgical blade of claim 9, wherein the cutting edge is curved.

15. The ultrasonic surgical blade of claim 9, wherein the distal end defines a beveled cutting surface.

16. An ultrasonic surgical blade, comprising:
a. a body having an external surface, a distal end movable relative to a longitudinal axis by vibrations applied thereto, and a cutting edge having a distal portion and a proximal portion, wherein at least a portion of the cutting edge comprises a sharp point and a beveled surface.

17. The ultrasonic surgical blade of claim 16, wherein the beveled surface and sharp point are located at the distal portion.

18. The ultrasonic surgical blade of claim 16, wherein the beveled surface and sharp point are located at the proximal portion.

19. The ultrasonic surgical blade of claim 16, wherein the blade comprises a second cutting edge having a distal portion and proximal portion, wherein at least a portion of the second cutting edge comprises a beveled surface.

20. The ultrasonic surgical blade of claim 16, wherein the cutting edge is straight.

21. The ultrasonic surgical blade of claim 16, wherein the cutting edge is curved.

22. The ultrasonic surgical blade of claim 19, wherein the second cutting edge comprises a sharp point.

23. The ultrasonic surgical blade of claim 16, wherein the distal end defines a beveled cutting surface.
